(19) United States
(12) Patent Application Publication
(10) Pub. No.: US 2025/0034336 A1
(43) Pub. Date: Jan. 30, 2025

(54) MICROGELS, METHODS OF MAKING MICROGELS AND METHODS OF USING MICROGELS

(71) Applicant: University of Florida Research Foundation, Inc., Gainesville, FL (US)

(72) Inventors: Wallace Gregory Sawyer, Gainesville, FL (US); Brent S. Sumerlin, Gainesville, FL (US); Duy T. Nguyen, Gainesville, FL (US); Jared Ian Bowman, Gainesville, FL (US)

(21) Appl. No.: 18/716,560

(22) PCT Filed: Dec. 16, 2022
(86) PCT No.: PCT/US2022/081792

§ 371 Date: Jun. 5, 2024

Related U.S. Application Data
(60) Provisional application No. 63/291,531, filed on Dec. 20, 2021.

(51) Int. Cl. C08J 3/075; A61K 47/69

(52) U.S. Cl. CPC C08J 3/075; A61K 47/6903; C08J 2333/02; C08J 2333/26

(57) ABSTRACT

The present disclosure provides for microgels, microgel compositions, methods of making microgels and microgel compositions, methods of using microgels and microgel compositions, and the like. The present disclosure provides for microgel compositions having a plurality of agent functionalized microgel particles that have a neutral charge. The agent functionalized microgel particles can be made of a polymer, where the polymer can be functionalized with an activated functional group that are quenched or bonded to at least one type of agent through the activated functional group. The agent functionalized microgel particles can have a longest dimension of about 5 to 150 μm and an aspect ratio of about 1 to 1.5.

### CLAIM OF PRIORITY TO RELATED APPLICATION

This application claims priority to co-pending U.S. provisional application entitled “MICROGELS, METHODS OF MAKING MICROGELS AND METHODS OF USING MICROGELS” having Ser. No. 63/291,531 filed on Dec. 20, 2021, which is entirely incorporated herein by reference.

### BACKGROUND

Microgels are widely used but many have drawbacks in regard to modifying the microgel with function groups. Also, microgels can be time consuming to produce and expensive. Therefore, there is a need to produce microgel with more options and using more efficient processes.

### SUMMARY

Aspects of the present disclosure provides for microgels, microgel compositions, methods of making microgels and microgel compositions, methods of using microgels and microgel compositions, and the like.

The present disclosure provides for microgel compositions comprising: a plurality of agent functionalized microgel particles that have a neutral charge, wherein the agent functionalized microgel particles are made of a polymer, wherein the polymer is functionalized with an activated functional group that are quenched or bonded to at least one type of agent through the activated functional group, wherein the agent functionalized microgel particles have a longest dimension of about 5 to 150 μm and an aspect ratio of about 1 to 1.5.

The present disclosure provides for microgel compositions as described above and herein where the activated functional group is an activated ester, N-acylbenzotriazole group, an anhydride group, or an activated carbonate functional group as well as ketone/aldehydes for Schiff/imine chemistry, or maleimides/acrylates for click chemistry.

The present disclosure provides for microgel compositions as described above and herein where the activated ester is selected from N-hydroxysuccinimide (NHS) ester group, sulfonated NHS ester group, fluorophenyl ester group, tosylate ester group, mesylate ester group, or O-acylisourea.

The present disclosure provides for microgel compositions as described above and herein where the activated carbonate functional group is a succinimidyl carbonate.

The present disclosure provides for microgel compositions as described above and herein where the agent functionalized microgel particles have an aspect ratio of about 1 to 1.5.

The present disclosure provides for microgel compositions as described above and herein where agent functionalized microgel particles have a random three-dimensional shape.

The present disclosure provides for microgel compositions as described above and herein where the polymer is selected from polyethylene glycol, polyacrylamide, polyacrylic acid, HEMA and combination thereof.

The present disclosure provides for microgel compositions as described above and herein where the polymer prior to bonding with the at least one type of agent has a concentration of an activated ester group of about 0.1 to 30 mol %.

The present disclosure provides for microgel compositions as described above and herein where about 90-95% or more of the at least one agent is on the surface of the agent functionalized microgel particles.

The present disclosure provides for microgel compositions as described above and herein where the agent is a protein.

The present disclosure provides for microgel compositions as described above and herein where the protein is an antibody, enzyme, or cytokine.

The present disclosure provides for microgel compositions as described above and herein where the agents are biological molecules containing amine groups.

The present disclosure provides for microgel compositions as described above and herein where the agent functionalized microgel particles is includes about 2 to 10 different types of agents.

The present disclosure provides for methods of making a composition of agent functionalized microgel particles as described above and herein comprising: forming a uniform distribution of the microgel particles, wherein the polymer microgel particles have activated functional groups; separating the uniform distribution of microgel particles using a centrifugation process to obtain a set of microgel particles, wherein the set of microgel particles has a longest dimension of about 5 to 150 μm and an aspect ratio of about 1 to 1.5; bonding an agent to a first amount of the activated functional groups of the polymer of the set of microgel particles, wherein a second amount of the activated functional groups are not bonded to the agent; and quenching the second amount set of agent functional groups with an amine group to form the agent functionalized microgel particles.

The present disclosure provides for methods of making a composition of agent functionalized microgel particles as described above and herein where the steps of forming, separating, bonding and quenching are processed over a time period of about 60 to 120 minutes.

The present disclosure provides for methods of making a composition of agent functionalized microgel particles as described above and herein where the agent functionalized microgel particle has a neutral charge, wherein the agent functionalized microgel particle is made of a polymer, wherein the polymer is functionalized with an activated functional group that are quenched or bonded to at least one type of agent through the activated functional group, wherein the agent functionalized microgel particles have a longest dimension of about 5 to 150 μm and an aspect ratio of about 1 to 1.5.

The present disclosure provides for methods of making a composition of agent functionalized microgel particles as described above and herein where the activated functional group is an activated ester, N-acylbenzotriazole group, an anhydride group, or an activated carbonate functional group as well as ketone/aldehydes for Schiff/imine chemistry, or maleimides/acrylates for click chemistry.

The present disclosure provides for methods of making a composition of agent functionalized microgel particles as described above and herein where the activated ester is selected from N-hydroxysuccinimide (NHS) ester group, sulfonated NHS ester group, fluorophenyl ester group, tosylate ester group, mesylate ester group, or O-acylisourea.

The present disclosure provides for methods of making a composition of agent functionalized microgel particles as described above and herein where the activated carbonate functional group is a succinimidyl carbonate.

The present disclosure provides for methods of making a composition of agent functionalized microgel particles as described above and herein where the agent functionalized microgel particles have an aspect ratio of about 1-1.5.

The present disclosure provides for methods of making a composition of agent functionalized microgel particles as described above and herein where agent functionalized microgel particles have a random three-dimensional shape.

The present disclosure provides for methods of making a composition of agent functionalized microgel particles as described above and herein where the polymer is selected from polyethylene glycol, polyacrylamide, polyacrylic acid, HEMA and combination thereof.

The present disclosure provides for methods of making a composition of agent functionalized microgel particles as described above and herein where the polymer prior to bonding with the at least one type of agent has a concentration of an activated ester group of about 0.1 to 30 mol %.

The present disclosure provides for methods of making a composition of agent functionalized microgel particles as described above and herein where about 90-95% or more of the at least one agent is on the surface of the agent functionalized microgel particles.

The present disclosure provides for methods of making a composition of agent functionalized microgel particles as described above and herein where the agent is a protein.

The present disclosure provides for methods of making a composition of agent functionalized microgel particles as described above and herein where the protein is an antibody, enzyme, or cytokine.

The present disclosure provides for methods of making a composition of agent functionalized microgel particles as described above and herein where the agents are biological molecules containing amine groups.

The present disclosure provides for methods of making a composition of agent functionalized microgel particles as described above and herein where the agent functionalized microgel particles is includes about 2 to 10 different types of agents.

The present disclosure provides for methods of making a composition of agent functionalized microgel particles, comprising: disposing a microgel block on a first side of a mechanical sieve, wherein the mechanical sieve has a selective characteristic size sieve, wherein the mechanic sieve has a second side on the side opposite the first side, wherein the microgel block includes polymers having activated functional groups; applying a first load against the microgel block to push the microgel block through the mechanical sieve to form a first plurality of fragmented pieces of the microgel block on the second side of the mechanical sieve, wherein a collection reservoir is positioned adjacent the second side of the mechanical sieve to receive the fragmented pieces of the microgel block; repeating this step using a new reservoirs until a uniform distribution of the microgel particles are formed; separating the uniform distribution of microgel particles using a centrifugation process to obtain a set of microgel particles, wherein the set of microgel particles having a longest dimension of about 5 to 150 μm and an aspect ratio of about 1 to 1.5; bonding an agent to a first amount of the activated functional groups of the polymer of the set of microgel particles, wherein a second amount of the activated functional groups are not bonded to the agent; and quenching the second amount of the agent functional groups with an amine group to form the composition of the agent functionalized microgel particles.

The present disclosure provides for methods of making a composition of agent functionalized microgel particles described above and herein where the steps of disposing the microgel block, applying, disposing the first plurality of fragmented pieces, separating, bonding and quenching are processed over a time period of about 60 to 120 minutes.

The present disclosure provides for methods of making a composition of agent functionalized microgel particles described above and herein where quenching includes using about 1.1 to 10 equivalents of the amine.

The present disclosure provides for methods of making a composition of agent functionalized microgel particles described above and herein where the amine is a primary amine.

The present disclosure provides for methods of making a composition of agent functionalized microgel particles described above and herein where quenching includes using an excess molar amount of the amine with respect to the second amount set of agent functional groups.

The present disclosure provides for methods of making a composition of agent functionalized microgel particles described above and herein where the load is a mechanical force.

The present disclosure provides for methods of making a composition of agent functionalized microgel particles described above and herein where the mechanical force is applied via a sterile syringe.

The present disclosure provides for methods of making a composition of agent functionalized microgel particles described above and herein where the mechanical sieve has a selective characteristic size sieve of about 10 to 85 micrometer mesh.

The present disclosure provides for methods of making a composition of agent functionalized microgel particles described above and herein where the first load is a uniformly distributed normal load.

The present disclosure provides for microgel polymers comprising a polymer is functionalized with a first concentration of unquenched activated functional groups.

The present disclosure provides for microgel polymers as described above and herein where the activated functional group is an activated ester, N-acylbenzotriazole group, an anhydride group, or an activated carbonate functional group as well as ketone/aldehydes for Schiff/imine chemistry, or maleimides/acrylates for click chemistry.

The present disclosure provides for microgel polymers as described above and herein where the activated ester is selected from N-hydroxysuccinimide (NHS) ester group, sulfonated NHS ester group, fluorophenyl ester group, tosylate ester group, mesylate ester group, or O-acylisourea.

The present disclosure provides for microgel polymers as described above and herein where the activated carbonate functional group is a succinimidyl carbonate.

The present disclosure provides for microgel polymers as described above and herein where the first concentration of the activated functional group of about 0.1 to 30 mol % or about 5 to 30 mol %.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 illustrates a method of coating the microgels with proteins, antibodies, amino acids, biologics, or any protein-based materials involves a series of fabrication steps.

FIG. 2A-2E illustrate surface functionalized and protein conjugated PAAm microgels. FIG. 2A illustrates Type I Collagen protein conjugated on the surface of microgels. FIG. 2B illustrates confocal images of microgels with fluorescent antibody conjugated on the surface. FIG. 2C illustrate fluorescent image of adherent cells binding on Collagen I conjugated microgels. The cells were stained with phalloidin (green) for actin and Hoechst 33342 (blue) for nuclei. FIG. 2D illustrates bright field microscope images of the same cells adhering on Collagen I conjugated microgels. FIG. 2E illustrates an image on the right is an illustration of surface functionalized microgels with different proteins and antibodies.

FIG. 3 illustrates protein conjugated polyacrylamide microgels to visualize cell migration in microgels. The top left are fluorescent images of microgels with type I collagen coated (green) on the surface and rhodamine B (red) polyacrylamide microgels to aid visualization during confocal microscopy. As an example, time-lapse images (0-116 h) shown here reveal how cells adhere on collagen I conjugated microgels and spread out while being cultured for 116 h demonstrating biocompatibility and successful conjugation of the proteins on the surfaces of the microgel particles.

FIG. 4 illustrates a confocal image of live astrocytes binding onto protein conjugated microgel particles. The cells were labeled with CellTracker™ Orange CMRA Dye (Cat. C34551) and imaged with Nikon A1R HD25 confocal microscope with high-definition Galvano scanner.

### DETAILED DESCRIPTION

Embodiments of the present disclosure provide for microgels, microgel compositions, methods of making microgels and microgel compositions, methods of using microgels and microgel compositions, and the like. Additional details are provided herein.

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, polymer chemistry, biochemistry, biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

“Polymers” are understood to include, but are not limited to, homopolymers, copolymers, such as for example, block, graft, random and alternating copolymers, terpolymers, etc. and blends and modifications thereof.

### DESCRIPTION

Embodiments of the present disclosure provide for microgels (e.g., functionalized microgels), microgel compositions including a plurality of agent functionalized microgel particles, methods of making microgels and microgel compositions, methods of using microgels and microgel compositions, and the like. In an embodiment, the present disclosure provides for agent functionalized microgel particles that comprise one or more types of agents bonded to the microgel particles. In an aspect, the present disclosure is advantageous in that the microgel compositions can be quickly produced (e.g., in under a few hours or hour) and consume a minimal amount of agent, which reduces costs. Also, the amount of agent(s) present on the agent functionalized microgel particles can be precisely controlled. The plurality of agent functionalized microgel particles can be used to study cell growth or deterioration, cell response to certain compounds, cell migration, differentiation, cell-cell interaction, and cell-extracellular matrix interaction.

The present disclosure provides for microgel polymers that include polymers that are functionalized with a first concentration of unquenched activated functional groups. The polymer can be polyethylene glycol, polyacrylic acid, polyacrylamide, hydroxyethylmethacrylate (HEMA), and copolymers thereof. The activated functional group can be selected from an activated ester (e.g., N-hydroxysuccinimide (NHS) ester group, sulfonated NHS ester group, fluorophenyl ester group, tosylate ester group, mesylate ester group, O-acylisourea ester), a N-acylbenzotriazole group, an anhydride group, or an activated carbonate functional groups (e.g., succinimidyl carbonate) as well as ketone/aldehydes for Schiff/imine chemistry, or maleimides/acrylates for click chemistry. The polymer can have a first concentration of the activated functional group of about 0.1 to 30 mol % or about 1 to 30 mol %, or about 5 to 30 mol %. In general, the microgel polymer can be made by mixing the monomers of interest to form the polymer and the activated functional group, where the time period to make the microgel polymer is about 20-30 minutes.

Additionally, microgel polymers functionalized with inactivated functional groups (e.g., carboxylic acid) can be prepared, and the functional groups can be later activated via formation of an activated ester (e.g., O-acylisourea ester or NHS ester) using 1-ethyl-3-(−3-dimethylaminopropyl) carbodiimide hydrochloride, for example.

The present disclosure also provides for microgel compositions, which can be made from the microgel polymer. The microgel composition includes a plurality of agent functionalized microgel particles that have a neutral charge. The agent functionalized microgel particles can be made of a polymer (e.g., polyethylene glycol, polyacrylamide, HEMA, polyacrylic acid) that is functionalized with an activated functional group (e.g., an activated ester, N-acylbenzotriazole group, an anhydride group, or an activated carbonate functional groups), where the activated functional group is quenched (e.g., reacted with an amine compound so the activated functional group is not hydrolyzed) or bonded to at least one type of agent through the activated functional group. The polymer of the agent functionalized microgel particles can have a concentration of the activated functional groups (e.g., quenched plus those bonded to agents) of about 0.1 to 30 mol % or about 1 to 30 mol %, or about 5 to 30 mol %.

In an aspect, the agent functionalized microgel particles have a longest dimension (e.g., diameter or width, length, or height) of about 5 to 150 μm and an aspect ratio of about 1 to 1.5, 1 to 1.2, or about 1. The agent functionalized microgel particles can have a random three-dimensional shape (e.g., an irregular shape). The agent functionalized microgel particles can be divided into subgroups with the dimensions described herein. For example, the longest dimension (e.g., diameter or width, length, or height) can have subgroups that are in a range of about 5 micrometers to 200 micrometers, about 5 micrometers to 150 micrometers, about 5 micrometers to 100 micrometers, about 10 micrometers to 200 micrometers, about 10 micrometers to 150 micrometers, about 10 micrometers to 100 micrometers, about 10 micrometers, about 100 micrometers, about 150 micrometers where the subgroup of the agent functionalized microgel particles can be selected based on the intended use, the cells of interest, the agents used, and the like.

In an embodiment, the agent can be a biological molecule containing amine groups (e.g., a primary amine). For example, the agent can be a protein, in particular, the agent can be an antibody, enzyme, cytokine, growth factors, or extracellular matrix components. In some embodiments it is desired for the agent functionalized microgel particles to include only one type of agent while in other embodiments it is desired for the agent functionalized microgel particles to include 2 or more types of agents (e.g., 2 to 10 or 2 to 5 or 2 to 3), for example two different types of antibodies or one antibody and one enzyme and the like. In an aspect, about 90% or more or about 95% or more of the at least one agent is on the surface of the microgel particles. The amount of the agent(s) on the agent functionalized microgel particles can be precisely controlled during the fabrication of the agent functionalized microgel particles by the amount of agent used in the fabrication method. In this way, the agent functionalized microgel particles can be made with a known amount of the agent(s) and made in a cost-effective manner with little or no waste of the agent. In this regard, about 10% to 100%, about 25% to 90%, about 40% to 80% of the activated functional group are each bonded to the agent.

Now having described the microgel polymer and the microgel compositions, methods of making the microgel composition is described below. In general, the methods of making a microgel composition including the agent functionalized microgel particles can include forming a uniform distribution of the microgel particles (described in more detail herein and below), where the microgel particles includes a polymer have activated functional groups. The uniform distribution of microgel particles can be separated using a centrifugation process to obtain a set of microgel particles, where the set of microgel particles has a longest dimension of about 5 to 150 μm or about and an aspect ratio of about 1 to 1.5 and 1.2 to 1.3 aspect ratio or subgroups therein. An agent can be bonded to a first amount of the activated functional groups (e.g., about 10% to 100%, about 25% to 90%, about 40% to 80% of the polymer) of the set of microgel particles and a second amount (e.g., less than 10%, about 10 to 75%, about 20 to 60%) of the activated functional groups are not bonded to the agent. The second amount set of agent functional groups can subsequently be quenched with an amine (e.g., ethanol amine). Quenching can include mixing an excess molar amount of the amine with respect to the second amount set of agent functional groups. In particular, quenching can include mixing about 1.1 to 10 or about 1.1 to 3, or about 2 equivalents of the amine (e.g., a primary amine such as ethanolamine, hexylamine, butylamine, and the like) with the microgel particles just after reacting the agent with the activated functional groups. In general, this process (e.g., separating, bonding the agent, and quenching) takes about 60 to 120 minutes.

Now having described the process in general, additional details are provided. In an aspect, the method of making a composition of agent functionalized microgel particles includes disposing a microgel block (e.g., made of the microgel polymer described herein) on a first side of a mechanical sieve. For example, a syringe can be used to dispose the microgel block on the mechanical sieve. The mechanical sieve has a selective characteristic size sieve (e.g., about 5 to 150 micrometer mesh, about 65 to 85 micrometer mesh, about 74 micrometer mesh) so that particles having the desired dimensions are formed. The mechanic sieve has a second side on the side opposite the first side, where the particles can be collected.

A first load (e.g., uniformly distributed normal load) can be applied against the microgel block to push the microgel block through the mechanical sieve to form a first plurality of fragmented pieces of the microgel block on the second side of the mechanical sieve. The first load can be provided the depression of the syringe to push the microgel block through the mesh of the sieve. The fragmented pieces of the microgel block can be received in a collection reservoir positioned adjacent the second side of the mechanical sieve. In general, this process can be repeated using the same mechanical sieve or using one or more other mechanical sieves having different mesh sizes to produce particles of the desired dimensions. For example, the first plurality of fragmented pieces of the microgel block can be optionally disposed (e.g., positioned, placed, etc.) on the first side of the mechanical sieve (e.g., the same sieve or another sieve with a different mesh size) and pushed through by applying a second load (e.g., using a syringe) against first plurality of fragmented pieces of the microgel block to push the fragmented pieces through the mechanical sieve to form a second plurality of fragmented pieces of the microgel block. The second plurality of fragmented pieces of the microgel block can be received in the collection reservoir. As needed, this process can be repeated until a uniform distribution (with a mean and standard deviation) of the microgel particles are formed. The more repeats of the process, the closer the normal distribution to the mean particle size.

As described above, the uniform distribution of microgel particles can be separated using centrifugation and then an agent(s) can be bonded to a portion of the activated functional groups while the remaining activated functional groups are quenched. This process (e.g., disposing step(s), applying step(s), separation, bonding, quenching) should take about 60 to 120 minutes to complete. For example, the process of gel polymerization with functional activated ester (e.g., N-hydroxysuccinimide (NHS) ester group) takes about 20-30 minutes, mechanical sieving for creation of gel particles takes about 5 minutes, separation to a certain particle size takes about 5 minutes, conjugation or bonding the gel particles with protein agents take about 30 minutes, quenching with ethanol-amine takes about 30 minutes, and total washing steps for the whole process take about 20 minutes.

The microgel composition produced including the agent functionalized microgel particles has a neutral charge.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

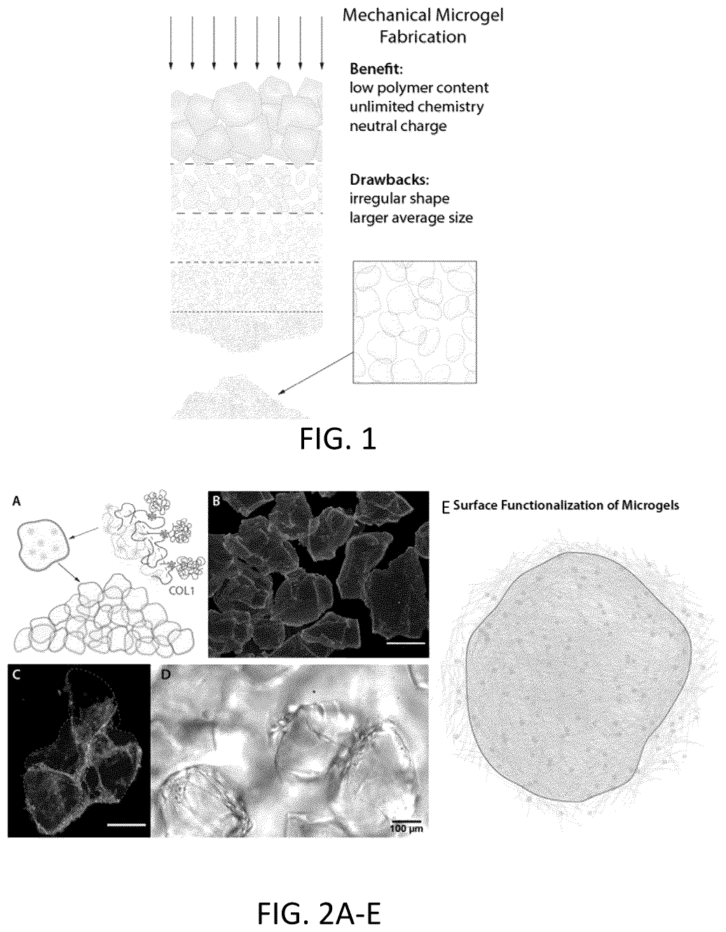

Mechanical fabrication of microgel particles allows for the rapid production of microgel particles with random and irregular shapes. FIG. 1 illustrates a method of coating the microgels with proteins, antibodies, amino acids, biologics, or any protein-based materials involves a series of fabrication steps.

1) Polymerization of a hydrogel material that contains a crosslinked concentration of NHS chemistry (N-Hydroxy-succinimide), for example.

2) After polymerization the hydrogel is crushed by passing it through a series of mechanical meshes or through the same mechanical mesh (not shown) until a final particle size distribution is produced. Generally we use between 2 micrometers and 1 mm sieve sizes.

3) After the production of the microgel, which still contains the NHS chemistry or similar chemistry, a dilute mixture of the candidate proteinaceous material is added to the aqueous mixture and gently agitated to allow for a uniform attachment on the surface. The penetration into the microgels of the materials depends on the concentration in the solution, the time of coating, and the mesh-size of the hydrogel material that the microgels are made from. Dilute solutions yield surface coverage as the surface is the first point of interaction, and the solution becomes depleted limiting coverage.

4) The remaining NHS chemistries are quenched using a high concentration of small molecules (e.g., an amine) that will diffuse into the hydrogel, react with the NHS, and prevent the NHS to hydrolyze to acrylic acid, for example.

5) Having produced a coated microgel ensemble, and quenching the remaining NHS in the microgel, the microgel ensemble is sorted into narrow size ranges for cell culture.

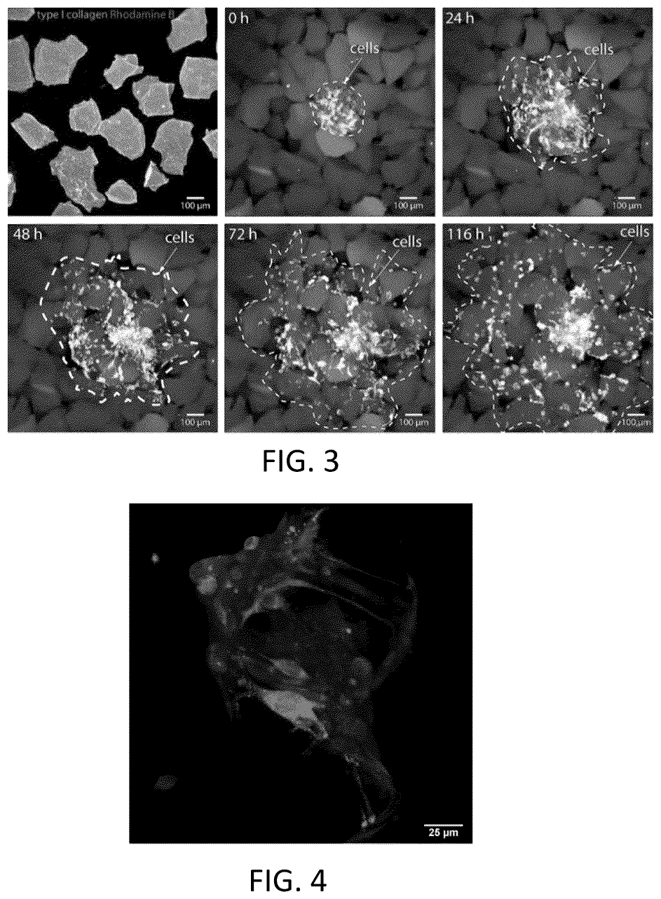

FIGS. 2A-1E illustrate surface functionalized and protein conjugated PAAm microgels. FIG. 3 illustrates protein conjugated polyacrylamide microgels to visualize cell migration in microgels. FIG. 4 illustrates a confocal image of live astrocytes binding onto protein conjugated microgel particles. Additional details regarding the figures are provided in the figure captions.

Ratios, concentrations, amounts, and other numerical data may be expressed in a range format. It is to be understood that such a range format is used for convenience and brevity, and should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figure of the numerical value. In addition, the phrase “about ‘x’ to ‘y’” includes “about ‘x’ to about ‘y’”.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A microgel composition comprising: a plurality of agent functionalized microgel particles that have a neutral charge, wherein the agent functionalized microgel particles are made of a polymer, wherein the polymer is functionalized with an activated functional group that are quenched or bonded to at least one type of agent through the activated functional group, wherein the agent functionalized microgel particles have a longest dimension of about 5 to 150 μm and an aspect ratio of about 1 to 1.5.

2. The microgel composition of claim 1, wherein the activated functional group is an activated ester, N-acylbenzotriazole group, an anhydride group, or an activated carbonate functional group.

3. The microgel composition of claim 2, wherein the activated ester is selected from N-hydroxysuccinimide (NHS) ester group, sulfonated NHS ester group, fluorophenyl ester group, tosylate ester group, mesylate ester group, or O-acylisourea.

4. The microgel composition of claim 2, wherein the activated carbonate functional group is a succinimidyl carbonate.

5. The microgel composition of claim 1, wherein the agent functionalized microgel particles have an aspect ratio of about 1.

6. The microgel composition of claim 1, wherein agent functionalized microgel particles have a random three-dimensional shape.

7. The microgel composition of claim 1, wherein the polymer is selected from polyethylene glycol, polyacrylamide, polyacrylic acid, HEMA and combination thereof.

8. The microgel composition of claim 1, wherein the polymer prior to bonding with the at least one type of agent has a concentration of an activated ester group of about 0.1 to 30 mol %.

9. The microgel composition of claim 1, wherein about 90% or more of the at least one agent is on the surface of the microgel particles.

10. The microgel composition of claim 1, wherein about 95% or more of the at least one agent is on the surface of the agent functionalized microgel particles.

11. The microgel composition of claim 1, wherein the agent is a protein.

12. The microgel composition of claim 11, wherein the protein is an antibody, enzyme, or cytokine.

13. The microgel composition of claim 1, wherein the agents are biological molecules containing amine groups.

14. The microgel composition of claim 1, wherein the agent functionalized microgel particles is includes about 2 to 10 different types of agents.

15. A method of making a composition of agent functionalized microgel particles, comprising:
forming a uniform distribution of the microgel particles, wherein the microgel particles include a polymer have activated functional groups;
separating the uniform distribution of microgel particles using a centrifugation process to obtain a set of microgel particles, wherein the set of microgel particles has a longest dimension of about 5 to 150 μm and an aspect ratio of about 1 to 1.5;
bonding an agent to a first amount of the activated functional groups of the polymer of the set of microgel particles, wherein a second amount of the activated functional groups are not bonded to the agent; and
quenching the second amount set of agent functional groups with an amine group to form the agent functionalized microgel particles.

16. The method of claim 15, wherein the steps of forming, separating, bonding and quenching are processed over a time period of about 60 to 120 minutes.

17. The method of claim 15, wherein the agent functionalized microgel particle has a neutral charge, wherein the agent functionalized microgel particle is made of a polymer, wherein the polymer is functionalized with an activated functional group that are quenched or bonded to at least one type of agent through the activated functional group, wherein the agent functionalized microgel particles have a longest dimension of about 5 to 150 μm and an aspect ratio of about 1 to 1.5.

18-29. (canceled)

30. A method of making a composition of agent functionalized microgel particles, comprising:
disposing a microgel block on a first side of a mechanical sieve, wherein the mechanical sieve has a selective characteristic size sieve, wherein the mechanic sieve has a second side on the side opposite the first side, wherein the microgel block includes polymers having activated functional groups;
applying a first load against the microgel block to push the microgel block through the mechanical sieve to form a first plurality of fragmented pieces of the microgel block on the second side of the mechanical sieve, wherein a collection reservoir is positioned adjacent the second side of the mechanical sieve to receive the fragmented pieces of the microgel block;
separating, using a new reservoir, the uniform distribution of microgel particles using a centrifugation process to obtain a set of microgel particles, wherein the set of microgel particles having a longest dimension of about 5 to 150 μm and an aspect ratio of about 1 to 1.5;
bonding an agent to a first amount of the activated functional groups of the polymer of the set of microgel particles, wherein a second amount of the activated functional groups are not bonded to the agent; and
quenching the second amount of the agent functional groups with an amine group to form the composition of the agent functionalized microgel particles.

31. The method of claim 30, wherein the steps of disposing the microgel block, applying, disposing the first plurality of fragmented pieces, separating, bonding and quenching are processed over a time period of about 60 to 120 minutes.

32. The method of claim 30, wherein quenching includes using about 1.1 to 10 equivalents of the amine.

33-45. (canceled)
